(19) United States
(12) Patent Application Publication
(10) Pub. No.: US 2007/0111211 A1
(43) Pub. Date: May 17, 2007

(54) Integrated viral complexes, methods of production thereof, vaccines containing same and methods of use thereof

(75) Inventor: Yair Saar, Kibbutz Merhavia (IL)

Correspondence Address: Kenneth I Kohn;Kohn & Associates, 30500 Northwestern Highway, Suite 410, Farmington Hills, MI, 48334, US

(73) Assignee: VIRUMAR PITUACH TARKIVIM LTD., ZEMACH (IL)

(21) Appl. No.: 10/585,058

(22) PCT Filed: Dec. 29, 2004
(86) PCT No.: PCT/IL04/01173

§ 371 Date: Jun. 30, 2006

Related U.S. Application Data
(60) Provisional application No. 60/532,886, filed on Dec. 30, 2003.

(51) Int. Cl. C12Q 1/68; C12P 21/06

(52) U.S. Cl. 435/6; 435/69.1

(57) ABSTRACT

Integrated viral complexes, methods of producing them, and their use as a vaccine against is described. Integrated viral complexes are optionally dried and may be stored at refrigeration temperatures. Integrated viral complexes include viable virions in intact cell membranes. The host cells are non-viable. The invention is advantageously employed in the context of Herpes virus including Marek's disease virus.

### FIELD AND BACKGROUND OF THE INVENTION

The present invention relates to integrated viral complexes, methods of production thereof, vaccines containing same and methods of use thereof and, more particularly, to integrated viral complexes of double stranded DNA virus, more particularly to integrated viral complexes of herpes virus such as Marek's Disease Virus (MDV).

Viruses are obligate intracellular parasites, that rely upon the nucleic acid replication and translation mechanisms of their host cell in order to accomplish their own replication. A complete infectious virus particle is called a virion. Viruses carry replication information stored as either DNA or RNA. Since viruses are obligate intracellular parasites and do not independently perform most metabolic functions associated with living cells, they are not susceptible to antibiotics. In most cases, treatment of viral infection is either inefficient or infeasible. Therefore, the classical, and still most effective, weapon in the war against viral diseases is the prevention of the disease by sanitation and immunization by vaccination.

Previously available vaccines can be broadly divided into three types: live vaccines, Inactivated (killed) vaccines, or genetically engineered (G.E.) vaccines (e.g. a recombinant subunit vaccine or Recombinant Vector Vaccines).

Live vaccines are sensitive biological substances that will lose their potency in time even when stored at suitable conditions. This loss of potency in live vaccines is correlated with the reduction of virus titer as vaccines are exposed to higher temperatures. The system used for keeping and distributing vaccines in good condition is called the ‘cold chain’. This consists of a series of storage and transport links, all of which are designed to keep the vaccine at suitable temperature until it reaches the user. A broken cold chain may cause inactivation of the vaccine or reduced shelf life and vaccine failure as a consequence. Lyophilisation otherwise known as Freeze drying, is a method used to maintain viability of live virus preparation at above freezing temperatures. This facilitates storage and transport. However freeze-drying vaccine preparations are available only for cell free vaccines. Moreover upon administration, cell free vaccines are susceptible to neutralizing antibodies such as maternal antibodies. On the other hand, viruses of cell associated nature as the herpes viridae (e.g. Vericella zoster or Mark's disease virus) are heat sensitive and are much less stable than others as the lyophylized cell free form. There are various types of vaccines that have not yet been satisfactorily stabilized by freeze drying. For example, live virus vaccines, such as those of measles, rubella, mumps and varicella. Some vaccines have to be stored and transported at very low or ultra low temperatures.

Marek's Disease (MD) is a naturally occurring most devastating disease of chickens. All poultry producers will inevitably suffer losses from MD. The Disease was initially described by Dr J. Marek in 1907. Until vaccination against MD was widely used, the disease was the main reason for economical losses in poultry industry throughout the world. The most commonly used vaccines for MD are cell associated vaccines that have to be maintained at about minus one hundred and ninety six degrees centigrade during the “cold chain” using liquid nitrogen and is being carefully thawed and diluted immediately before use.

Marek's disease viruses are classified by serological techniques into three serotypes. The serotypes differ in their biological properties as well.

MDV serotype I (MDV-I) are usually tumorogenic and pathogenic in chickens. Attenuated strains of this serotype are available as cell associated vaccines. All strains of MDV-I remains highly cell associated so that cell free infectious virion is difficult to obtain except as dander from feather follicles.

MDV serotype II are naturally occurring non virulent viruses. MDV-II vaccines are available as cell associated preparation.

MDV serotype III is herpes virus of turkeys (HVT). It is apathogenic in turkeys and chickens, however it is antigenically related to the first two serotypes and can be used for vaccination against MD in chickens.

Unlike MDV serotypes I or II vaccines, HVT derived vaccines against MD, can be produced as either cell free lyophilized vaccine or cell associated frozen vaccines. However, this strain of vaccine is of very low efficacy especially as cell free. Since the end of 1970's there are growing number of outbursts of MD. Viruses from these outbursts were shown, under controlled condition, to be pathogenic to HVT vaccinated chickens. The emergence of very virulent MDV-I (vvMDV-I) was reported from different parts of the world. It is possible that vvMDV-I may replicate in vaccinated chickens. Inadequate vaccination may be a selective force favoring evolution of more virulent strains. Vaccines containing more than one serotype may be useful in controlling the disease caused by vvMDV.

Serotype 1 and serotype 2 vaccines have to be stored and administered as cell-associated preparations since sufficient yield of cell free viruses can only be extracted from HVT infected cells. (Powell, P. C., World's Poultry Science Journal 42, 205, 1986; Witter, R. L. et al , Avian Diseases 31, 829, 1987; Schat, K. A., Internews 3, 13, 1989). In practice, this means that storage and transportation of said vaccines have to take place in liquid nitrogen at about (−)196 degrees C. Cell associated vaccines are therefore, more expensive and more difficult to handle. Proper handling of vaccine during thawing and reconstitution is crucial. (Halvorson, D. A. and D. O. Mitchell, Loss of cell-associated Mark's disease vaccine titer during thawing, reconstitution and use. 1979, Avian Dis 23:848-853). In some countries their use is not applicable. Moreover, these vaccines are susceptible to mishandling and physical abuse. Before use, ampules of cell associated vaccine should be carefully thawed and diluted otherwise incorrect dose will be administered.

Two United States Patents issued to Skinner (U.S. Pat. Nos. 4,816,250 and 5,219,567) teach methods for preparing vaccines from herpes viruses and vaccines which protect against herpes virus infection. Skinner's teachings contain as in inherent limitation a requirement for disruption of the host cell membrane. Skinner teaches isolation of viral components from the cytoplasm of killed host cells. Sub-unit vaccines of this type have, as an inherent disadvantage, a low efficacy relative to whole virus preparations.

There is thus a widely recognized need for, and it would be highly advantageous to have, integrated viral complexes, vaccines containing same and methods of use thereof devoid of the above limitation(s).

### SUMMARY OF THE INVENTION

According to one aspect of the present invention there is provided an integrated viral complex. The complex includes: (a) a plurality of intact cell membranes, each of the intact cell membranes belonging to a non-viable cell; and (b) a plurality of viable virions. A majority of the virions of the plurality of viable virions are contained within the intact cell membrane belonging to the plurality of intact cell membranes. In layman's terms, the intact cell membranes “cocoon” the virions.

According to another aspect of the present invention there is provided a pharmaceutical composition for vaccination. The pharmaceutical composition includes: (a) a plurality of intact cell membranes, each of the intact cell membranes belonging to a non-viable cell; and (b) a plurality of viable virions, a majority of the virions of the plurality of viable virions contained within the intact cell membrane belonging to the plurality of intact cell membranes; and (c) carriers and excipients.

According to yet another aspect of the present invention there is provided a method for producing integrated viral complexes, the method includes: (a) growing a population of individual cells in culture; (b) infecting the individual cells belonging to the population with an aliquot of viable virions so that a desired viral yield is achieved; (c) harvesting the population of individual cells characterized by the desired viral yield to a storage medium containing a cryoprotectant; (d) storing the population of individual cells characterized by the desired viral yield at a temperature in the range of (−) 30 to (+) 8 degrees centigrade.

According to still another aspect of the present invention there is provided a method of producing a pharmaceutical composition for vaccination. The method includes: (a) growing a population of individual cells in culture; (b) infecting the individual cells belonging to the population with an aliquot of viable virions so that a desired viral yield is achieved; (c) harvesting the population of individual cells characterized by the desired viral yield to a storage medium containing a cryoprotectant; (d) dividing the population of individual cells characterized by the desired viral yield into dosage portions suited for vaccination of a specified number of subjects; and (e) storing the dosage portions at a temperature in the range of (−) 30 to (+)8 degrees centigrade.

According to an additional aspect of the present invention there is provided a method of vaccination which employs an integrated viral complex, the method includes administering to a subject at least one dose of an amount of an integrated viral complex sufficient to elicit an active immune response in a subject.

According to further features in preferred embodiments of the invention described below, the virions are DNA virions, more preferably double stranded DNA virions, most preferably the double stranded DNA virions belong to the herpes viruses, for example Marek's disease virus.

According to still further features in the described preferred embodiments the pharmaceutical composition is supplied as an article of manufacture including packaging material and instructions for use.

According to still further features in the described preferred embodiments the infecting employ a viral preparation selected from the group consisting of a cell free preparation and a cell associated preparation.

According to still further features in the described preferred embodiments the cryoprotectant includes at least one material selected from the group consisting of glycerol, DMSO, and sugars.

According to still further features in the described preferred embodiments the desired yield is in the range of 0.001 to 1 PFU/cell.

According to still further features in the described preferred embodiments the temperature is in the range of (+) 2 to (+) 8 degrees centigrade.

According to still further features in the described preferred embodiments the method further includes passaging the individual cells belonging to the population with the desired viral yield as means of increasing a total output of virions.

According to still further features in the described preferred embodiments the method further includes reducing a volume of the storage medium so that a desired number of cells per unit volume is achieved.

According to still further features in the described preferred embodiments method further includes drying the population of individual cells.

According to still further features in the described preferred embodiments the dosage portions each individually include a number of doses in the range of 1 to 1 million.

According to still further features in the described preferred embodiments the subject is a member of an avian species.

According to still further features in the described preferred embodiments the administration is conducted in ovo.

According to still further features in the described preferred embodiments the administration is conducted via IM injection at 1 day of age.

According to still further features in the described preferred embodiments the administration is conducted via subcutaneous injection.

According to still further features in the described preferred embodiments the administration is conducted via aerosol spray.

The present invention successfully addresses the shortcomings of the presently known configurations by providing an integrated viral complex, methods of manufacture thereof, vaccines including same and methods of use thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified flow diagram illustrating events associated with performance of methods according to the present invention.

### DESCRIPTION OF THE PREFERRED EMBODIMENTS

The present invention is of an integrated viral complex, methods of production thereof, vaccines containing same and methods of use thereof and, more particularly, to integrated viral complexes of double stranded DNA viruses.

For purposes of this specification and the accompanying claims, the phrase “integrated viral complex” refers to live virions contained, or cocooned, in host cell membranes with a reduced cytosolic content.

The present invention finds especial utility in the context of Herpes viruses including, but not limited to herpes viruses of medical and veterinary importance, such as Marek's Disease Virus, Herpes Simplex (oral and genital herpes in humans,) Herpes varicella, ILTV-like viruses, Cytomegalovirus, Muromegalovirus, Roseolovirus, Lymphocryptovirus, Rhadinovirus and Ictalurid herpes-like viruses.

Use of the present invention in the context of a Marek's disease vaccine based upon integrated viral complexes is described in detail in example set forth hereinbelow. These examples make it abundantly clear that the present invention can be used for vaccination of commercial poultry flocks.

According to one aspect of the present invention there is provided an integrated viral complex. The complex includes a plurality of intact cell membranes belonging to non-viable cells and a plurality of viable virions. A majority of the virions are contained within the intact cell membranes.

The integrated viral complexes are optionally, but preferably, provided as a pharmaceurical composition for vaccination which further includes carriers, stabilizers and excipients.

As used herein a “pharmaceutical composition” refers to a preparation of one or more of the active ingredients described herein with other chemical components such as physiologically suitable carriers, stabilizers and excipients. The purpose of a pharmaceutical composition is to facilitate stability of the vaccine and administration of a compound to an organism.

Herein the term “active ingredient” refers to the integrated viral complex accountable for the biological effect.

Hereinafter, the term “carrier” refers to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Techniques for formulation and administration of vaccines are summarized in the Oie vaccine manual and Eu Phar which are which are incorporated herein by reference According to further features in preferred embodiments of the invention described below, the virions are DNA virions, more preferably double stranded DNA virions, most preferably the double stranded DNA-virions belong to the herpes viruses, for example Marek's disease virus virions.

According to still further features in the described preferred embodiments the pharmaceutical composition is supplied as an article of manufacture including packaging material and instructions for use.

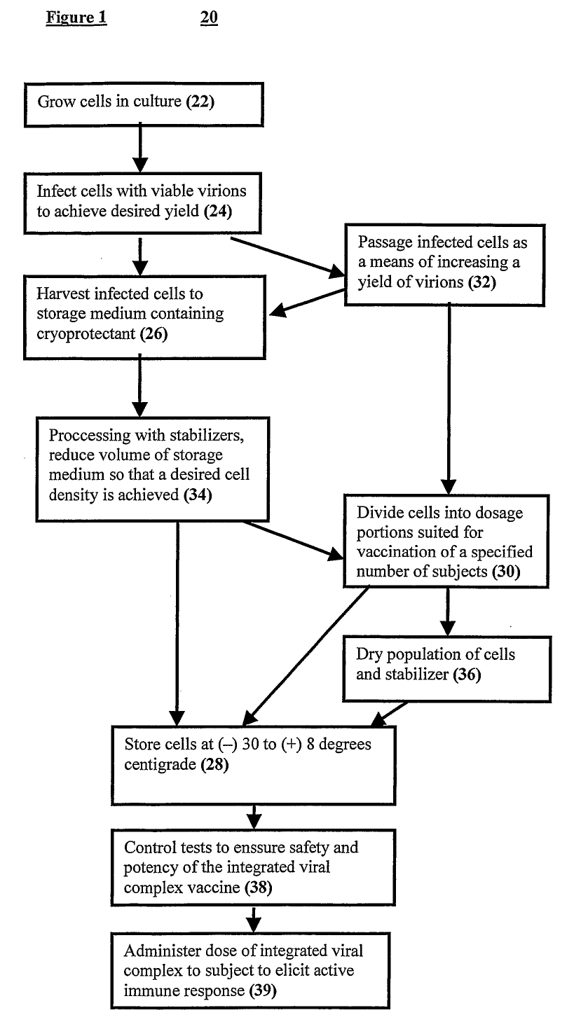

According to yet another aspect of the present invention there is provided a method 20 (FIG. 1) for producing integrated viral complexes. Method 20 includes growing 22 a population of individual cells in culture. Method 20 further includes infecting 24 the individual cells belonging to the population with an aliquot of viable virions so that a desired viral yield is achieved. For purposes of this specification and the accompanying claims, “viral yield” refers to the average number of PFU/cell at the time of harvest of cells from culture for preparation of integrated viral complexes. Method 20 further includes transferring 26 the population of individual cells characterized by the desired viral yield to a storage medium containing a cryoprotectant. Transferring 26 may be accomplished by centrifugation and re-suspension or by addition of ingredients to a culture medium or a combination thereof and thus may serve as a means of harvest. Method 20 further includes storing 28 the population of individual cells characterized by the desired viral yield at a temperature in the range of (−) 30 to (+) 8 degrees centigrade.

Preferably, method 20 is a method of producing a pharmaceutical composition for vaccination and further includes dividing 30 the population of individual cells characterized by the desired viral yield into dosage portions suited for vaccination of a specified number of subjects.

Method 20 further includes method of vaccination 39 which employs an integrated viral complex and includes administering to a subject at least one dose of an amount of an integrated viral complex sufficient to elicit an active immune response in the subject.

According various preferred embodiments of method 20, infecting 24 may employ a a cell free preparation or a cell associated preparation of virions.

According to still further features of the invention, the cryoprotectant may include for example glycerol, DMSO, and sugars as detailed hereinbelow.

Briefly, glycerol is preferably employed at 2-8% v/v achieved via stepwise addition. Sodium glutamate is preferably employed at from 0.1 to about 2% (w/v), sucrose at from about 1.0 to about 7.5% (w/v), and hydrolyzed gelatin from about 0.5 to about 5% (w/v),

According to still further features in the described preferred embodiments the desired viral yield is in the range of 0.001 to 1 PFU/cell.

According to a most preferred embodiment of the invention, the integrated viral complexes produced by method 20 are stable for long periods of time at temperatures in the range of (+) 2 to (+) 8 degrees centigrade.

Optionally, but preferably, method 20 further includes passaging 32 the individual infected cells belonging to the population with the desired viral yield to a fresh cell culture as a means of increasing a yield of the virions.

Optionally, but preferably, method 20 further includes reducing 34 a volume of the storage medium so that a desired number of cells per unit volume is achieved.

Optionally, but preferably, method 20 further includes drying 36 the population of individual cells. Drying may be, for example, by lyophilization.

Optionally, but preferably, method 20 is conducted in the context of standard quality control testing 38 of the vaccine. Tests may be, for example, purity tests, safety tests or potency test.

Dosage portions may contain a number of doses in the range of 1 to 1 million. For human medicine, single dose dosage portions are preferred. For veterinary use, especially in the context of commercial poultry production, large dosage portions are preferred.

Optionally, but preferably, the subject to be vaccinated is a member of an avian species such as a chicken, a turkey, a goose, a duck a mallard or a berber.

In the context of vaccination of avian subjects, administration of at least one dose of a vaccine is preferably in ovo, at 18 days of incubation, or via IM or SC injection, or spray methods from 1 day of age. This practice prevents unwanted exposure to relevant pathogens prior to vaccination.

The principles and operation of integrated viral complexes, methods of production thereof, vaccines containing same and methods of use thereof according to the present invention may be better understood with reference to the accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present invention provides, for the first time, a live vaccine based upon integrated viral complexes. Integrated viral complexes may be stored at standard refrigerating or freezing temperatures as demonstrated in examples 1 and 3 hereinbelow. This is an inherent advantage with respect to previously known cell-associated or cell free vaccines. In other words, integrated viral complexes according to the present invention offer the ease of storage and handling previously associated with lyophilized vaccines and the efficacy of cell-associated live vaccines. This makes the present invention suitable for use in places where storage in liquid nitrogen is not feasible. Alternately, or additionally, it reduces storage costs of large amounts of vaccine produced on an industrial scale.

Further, the present invention is not subject to interference from maternal antibodies as illustrated in example 2 hereinbelow. This makes it vastly superior to previously available cell free vaccines, especially in in ovo inoculation or injection (e.g. IM) in one day old chicks.

According to one preferred embodiment of the present invention a vaccine is provided for the protection of poultry against MD, characterized in that this vaccine comprises infectious vaccine's MD serotype 1, 2 or 3 viruses enclosed in a cell membrane together with a pharmaceutically acceptable stabilizer.

Briefly, and by way of illustrative example, an integrated viral complex according to the invention can be prepared by serial expansion of a selected virus (e.g. MDV serotype 1 (CV1988), 2 (SB1) or 3 (IVT-FC126)) of acceptable passage number in an appropriate cell culture, harvesting the host cells with a desired viral yield and processing the infected host cells to produce integrated viral complexes with viable virions contained in essentially intact cell membranes of non-viable host cells. These integrated viral complexes are suitable for use as vaccines as demonstrated hereinbelow in Examples 1, 4 and 5.

An integrated viral complex based vaccine according to the present invention can be derived from any serotype 1 MD virus strain, such as for example the CV1988 strain, or from any serotype 2 MD virus strain, such as for example the SB-1 strain (Schat et al., U.S. Pat. No. 4,160,024; commercial available from Cornell University.), or from any serotype 3 MD virus strain, such as for example the HVT FC126 strain.

Serial passaging and expansion of the serotype 1 MD-viruses can accomplished by methods known in the art for this purpose. Briefly, viruses are grown in a suitable cell culture; harvested infected cells are inoculated to a medium containing a fresh cell culture. The serotype 1 MD viruses are subjected to several serial passages in cell culture until a usable quantity of virus can be obtained within the cell, and thereafter processed into a vaccine.

Suitable cell cultures for serial passage include, but are not limited to, chick kidney (CK), chicken embryo fibroblast (CEF) and chicken embryo skin cultures (CESC).

More in particular, serotype 1 MD viruses can be seeded onto 24 to 48 hour monolayers of CK, CEF or CESC cultures which are then maintained for several days at 37 degrees C. The contents of a suitable growth medium are for example: Eagles basal medium (BME), Medium 199,F10, Dulbecco's Modified Eagle Medium (DMEM), F12, sodium bicarbonate, Bovine fetal serum and antibiotics. Cells are passaged when 75% or more of the monolayer is cytopathically affected. At the end of the incubation period, the whole mass of cells is washed with phosphate-buffered saline, dispersed with trypsin with EDTA and resuspended in a small amount of culture medium, and replated and grown on fresh monolayer cell cultures as described above. The number of subsequent passages is dependent of the quantity of virus obtainable from the culture and of the preservation of the immunogenic and infectious properties of the passaged virus. Cells of the last passage can be washed, trypsinized, centrifuged and dispersed in a small volume of culture medium containing dimethyl sulfoxide (DMSO). This preparation can be slow-frozen to (−)70 degrees C. and transferred to liquid nitrogen temperatures to be used as seed virus culture.

Typically, integrated viral complex vaccine preparations can be obtained according to the method described above which have a titer ranging from 300 to 2500 PFU per dose.

The number of passages which are necessary to obtain serotype 1, 2 or 3 MD viruses which yield sufficient amounts of infectious virus is dependent on the specific serotype 1, 2 or 3 MD strain and its passages level the desired quantity of virus titer.

Subsequently, to propagate the serotype 1, 2 or 3 MD viruses, roller bottle cultures seeded with cells such as CEF can be inoculated with cell-associated or cell-free virus obtained as described above after 24-72 hours of incubation. After a further incubation period of several days the supernatant medium is discarded and the cells removed with a trypsin EDTA mixture. The cells can be then harvested by centrifugation and the supernatant is discarded.

In order to prepare the integrated viral complex based vaccine preparations the deposited cells can be suspended in stabilizer containing cell media, for example M199 with solution of EDTA trehalose, glycerol, sucrose, gelatin alginate salts. Cell dehydration may be effectuated by several methods, e.g. freeze drying or vacuum drying. The presence and the number of intact cells can be observed upon examination in a hemocytometer. The vaccine bulk preparation can be filled out in vials and can be dried if desired by freeze drying. The integrated viral complex based vaccine obtainable from the method described above can be incorporated in vaccines as live viruses or as inactivated viruses.

The vaccines containing live virus can be prepared and marketed in the form of a suspension, dehydrated or lyophilized.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include virological, molecular, biochemical, microbiological and cell cultures techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Animal Cell Culture” Freshney, R. I., ed. (1986); “Immobilized Cells and Enzymes” IRL Press, (1986); Mowat, N. and M. Rweyemamu, Eds. (1997). Vaccine Manual The production and quality control of veterinary vaccines for use in developing countries. FAO Animal Production and Health Series No 35. Rome, Food and Agriculture Organization of the United Nations.

All of the above are incorporated by reference as if fully set forth herein. Other general references are provided throughout this document. The procedures therein are believed to be well known in the art and are provided for the convenience of the reader. All the information contained therein is incorporated herein by reference.

Before presenting examples, reference is made to the following materials and methods employed in performance of experiments described in the examples.

Materials and Methods:

Seed virus strains: CV1988: low passage MD CVI988 virus was obtained from Shafit biological laboratories, Israel, originally obtained from Cornell University, USA The vaccine master seed is the 18 passages of the original virus, passaged in SPF embryo skin cultures. After preparation of integrated viral complexes as detailed hereinbelow, the CVI988 integrated viral complex based vaccine is stored at standard refrigerator temperature (2 to 8 degrees C.).

HVT: Freeze dried vaccine—the virus origin is a low passage HVT viruses obtained from Cornell University, USA. The vaccine master seed is the 6 passages of the original virus, passaged in SPF embryo skin cultures.

After preparation of integrated viral complexes as detailed hereinabove, the HVT integrated viral complex based vaccine is stored in refrigerator temperature (2-8 degrees C.).

Challenge Virus: Viral challenge was with WMDV, a field isolate of very virulent case of vaccinated Layers flock in Israel designated as BE703. The virus was obtained from Kimron Veterinary Institute, Beit Dagan, the veterinary services of Israel.

Cell Associated (CA) vaccine CVI988: Rismavac™, Cell Associated (CA) CV1988 MD vaccine (Intervet, Holland) was purchased and stored in liquid Nitrogen.

Cell Associated (CA) HVT Vaccine: Marexine™-CA, Cell Associated (CA) HVT MD vaccine (Intervet, Holland) was purchased and stored in liquid Nitrogen.

Cell Free HVT Vaccine: The Cell Free HVT vaccine produced in Virumar as described at 1970 (Calnek, B. W et al. 1970. Lyophilization of cell free Mark's Disease herpesvirus and a herpesvirus from turkeys. App Microbiol 20:723-726). The vaccine was stored in Refrigarator temperature (2-8° C.).

Tissue culture media Standard culture medium such as M199-earle base, Eagle MEM (E-MEM), Dulbecco MEM (DMEM), F-10 or F-12 etc. were purchased from (Biological Industries Ltd.; Kibbutz Beit-Haemek, Israel). Culture media are optionally supplemented with amino acids, salts, anti-fungal or anti-bacterial agents, animal serum other additives according to accepted practice.

SPF Fertilized eggs: Embryonated eggs from Specific Pathogen Free (SPF) white Leghorn chickens flock were obtained commercially by Vireo Ltd, Israel from LAH, Germany (valo eggs). Within an experiment, eggs from a single flock were employed.

Chickens: White Leghorn breeder chickens for layer flocks were obtained commercially from Hassollellim Hatcheries, Israel. The chicks were raised in Positive pressure Isolatores. Within an experiment, chicks hatched from eggs from a single flock were employed. These chicks are not SPF and have normal titers of maternal antibodies. Isolators are employed to prevent uncontrolled exposure to MDV during vaccination/challenge experiments.

Tissue culture For MDV integrated viral complexes production, avian-derived culture cells are used. While chicken embryo fibroblast cells (CEF cells) are a most preferred host cell however, any cell culture may be employed so long as it will support growth of the relevant viral strain and it is free of viral contaminants.

Such a cell culture may be Duck Embryo Fibroblast cells (DEF), Chicken Embryo-derived cell line CHCC-OU2 (Ogura, H. et al., Acta Med Okayama 41(3): 141-143, 1987, and Coussens et al., Japanese Patent Publication No. 9-173059), quail-derived cell strain QT-35 (Spijkers et al., Japanese Patent Publication No. 9-98778) and other known avian cell culture systems may be employed without significantly altering the present invention.

Preparation of Integrated Viral Complexes: MDV CVI988

The MD virus infected cells were subjected to several serial passages onto a fresh cell culture until a usable quantity of cell integrated virus can be obtained there from, and thereafter processed into a vaccine.

This process for stabilizing the cell integrated viral complexes comprises of 4 preparation steps as follows:

(1) Preparation of sterile stabilizer solutions A and B.

(2) Preparation of a virus infected cell suspension.

(3) Preparation of a stabilized cell integrated live vaccine in which cryoprotectant added by dose to cultured cells.

(4) distribution of the vaccine bulk to containers or vials with lyophilization.

More in particular, MD virus was cultured by the use of a Chicken Embryo Fibroblast (CEF) cell culture. The CEF was derived from SPF fertilized eggs. The obtained culture is harvested, for example, by centrifugation (500 g), to thereby obtain a MD virus infected cells fraction. The Master Seed Virus CVI988 strain has been seeded onto 24 to 48 hour monolayer of CEF cultures which are then maintained for 24-72 hours at 37 degree C. The contents of a growth medium was: 50% Medium M-199 (Eagles), +L-glutamine, 50% Nutrient Mixture F-10, 5% Bovine fetal serum, sodium bicarbonate, and antibiotics (Biological Industries Ltd, Kibbutz Beit Haemek, Israel). Subsequently, to propagate the attenuated MD viruses, roller cultures seeded with CEF cells has been inoculated with cell-associated seed virus obtained as described above after 36 hours of incubation. After a further incubation period of 96 hours the supernatant medium was discarded and the cells harvested with a trypsin EDTA mixture where after the cells has been deposited by centrifugation (500 g) and the supernatant was discarded. Virus was propagated when 75% or more of the monolayer was cytopathically affected. At the end of the incubation period, the whole mass of cells were washed with phosphate-buffered saline, dispersed with trypsin and resuspended in a small amount of culture medium. To the infected cells fraction, a solution A, containing Medium M199 with glycerol 50% was added at the amount of 10% V/V of the total volume, every 15 nilnute until a soloution of 2-8% v/v of glycerol in cell suspension was obtained. Subsequently, the infected cells were collected by means of a centrifuge (500 g) and the supernatant was discarded. Solution B, a sterile stabilizer was added to the collected infected cells to thereby suspend the infected cells at a concentration of 5-30 million cells per ml.

The sugar(s) and cryoprotectant serve to osmotically deplete the cytoplasmic content of the host cells while leaving the host cell membrane intact. The stabilizer comprising from about 0.1 to about 2% (w/v) of sodium glutamate, from about 1.0 to about 7.5% (w/v) of sucrose, from about 0.5 to about 5% (w/v) of hydrolyzed gelatin, and from about 2.0 to about 8% (v/v) of Glycerol, all in a PBS soloution in terms of the concentration in the vaccine bulk.

The resultant integrated viral complexes the aliquoted into suitable containers (e.g. to glass vial with rubber stopper) and freeze dried. It is stressed that integrated viral complexes according to the present invention may be prepared from identically modified or recombinant virus as well as from naturally occurring field isolates.

Titration method in Tissue culture: Secondary chicken embryo fibroblasts (CEF) were preferably employed for viral titration. The titer of the commercial Cell Associated (CA) CVI988 vaccine (Rismavac™) was carried out following APHIS Supplemental Assay Method (SAM) No PYSAM0405.01 for titration of Mark's Disease Cell Associated Vaccine.

The titer of Rispred™—our Cell Integrated (CI) CVI988 vaccine was carried out following as detailed in the above mentioned SAM of APHIS USDA for lyophilized vaccine, with a modification of sonication with 1-1.5 Watt/ml of the inoculated dilution. The PFU were counted and PFU per vial was calculated. For each titration 5 replicates plates were tested.

### Example 1

### Stability of Integrated Viral complexes Under Adverse Storage Conditions

In order to establish the storage characteristics of integrated viral complexes according to the present invention, the titer of experimental batches of freeze dried cell integrated viral complex based CVI988 vaccine was tested after acceleration of storage conditions.

The titration of the integrated viral complex based CVI988 vaccine was measured as Plaque Forming Units (PFU) after storage at 2-8 degrees C., or after acceleration of storage conditions by incubation at 37 degrees C. for 3 days. The experimental vaccine was tested against commercial Rismavac™ (Intervet, the Netherlands) stored in liquid nitrogen. For each assayed product three vials were tested and 5 replicate plates from each vial were prepared.

Titration was performed as described hereinabove in materials and methods. Results are summarized in table 1.

TABLE 1
Effect of storage conditions on viral titre

| | | PFU per vial | |
|---|---|---|---|
| | PFU per vial | (×1000) after 7 d | PFU per vial |
| Type of | (×1000) before | storage at | (×1000) after |
| MD Vaccine | Acceleration | 2-8° C. | Acceleration |
| Cell Associated¹ | 1235 | 0 | 0 |
| integrated viral | 457 | 462 | 389 |
| complex² | | | |

¹commercially available, Rismavac ™
²according to the present invention

In summary, the results presented in table 1 indicate that the previously available cell associated MDV vaccine deteriorates completely in 7 days at standard refrigeration temperatures. In sharp contrast, the integrated viral complexes of the present invention retained a significant titer after 7 days standard refrigeration temperatures. Further, this titer was retained after an addition 3 days incubation at 37 degrees C.

### Example 2

### Comparative Efficacy of Integrated Viral Complex Based Vaccine and Cell Associated Vaccine in Commercial Chickens

In order to demonstrate the efficacy of integrated viral complexes according to the present invention three hundred non-SPF (with maternal antibodies) 1-day-old chicks (Hassollellim Hatcheries, Israel) were employed in a vaccination experiment with the CVI988 strain of MD and subjected to subsequent challenge.

The chicks were divided into three equal groups and vaccinated by IM injection at one day of age. Group a was vaccinated with 1000 PFU of Rismavac cell associated vaccine (Intervet, Holland). Group b was vaccinated with 1000 PFU of integrated viral complex based vaccine according to the present invention. Group c was injected with diluent only and served as a negative control.

It is important to note that the freeze dried integrated viral complex based vaccine was stored at four degrees centigrade while the commercially available cell associated vaccine required storage in liquid nitrogen until dilution immediately prior to use.

Challenge was administered at 9 days post vaccination as detailed herein above in materials and methods. The chickens were observed for 12 weeks post challenge (dpc). Mortality was recorded and each dead bird was subject to post mortem examination to ascertain presence or absence of specific macroscopic lesions of Marek's disease. At the end of the observation period the survivors were sacrificed and subject to post mortem examination to ascertain presence or absence of specific macroscopic lesions of Marek's disease. Birds with specific macroscopic lesions of Marek's Disease, were deemed to be MD positive. Results are summarized in Table 2.

Protection rates of the integrated viral complex based vaccine were similar to those of the commercial Cell Associated (CA) CVI988 vaccine (Rismavac) and obviated the need for liquid nitrogen storage.

TABLE 2
protective effect of 1000 PFU of integrated viral
complex and cell associated vaccine

| | | | # MD | |
|---|---|---|---|---|
| | | Storage | Positive/ | % |
| group | Vaccine type | temperatures | Total | Protection |
| a | Cell associated | (−)196 | 8/94 | 91.5 |
| b | Integrated viral complex | (+)2-8° C. | 9/96 | 93.7 |
| c | Unvaccinated control | NA | 86/95 | 14.7 |

### Example 3

### Stability of Integrated Viral Complex Based Vaccine Over Time at Refrigeration Temperatures

Results presented in Example 1 indicate integrated viral complexes according to the present invention are stable for short periods of time at 2-8 degrees centigrade. A commercially available cell associated vaccine as described earlier lost all activity under the same conditions. In order to establish that an integrated viral complex based vaccine according to the present invention is stable for a period of up to one year under standard refrigeration conditions, the titer of two experimental batches of Freeze Dried integrated viral complex CVI988 MD vaccine was assayed.

Titer of the integrated viral complex CVI988 MD vaccine virus was measured as Plaque Forming Units (PFU), using a modified titration method as described hereinabove in materials and methods. The vaccine was stored at 2-8° C. after production. Titrations were carried out after one, seven, and twelve months of storage. Results, summarized in table 3, indicate that the titer of the virus remained stable throughout the year. As illustrated in example 1, cell associated serotype 1 vaccines are completely degraded after 1 day under similar conditions. Results presented in tables 1 and 3 indicate that significant reduction of storage costs may be achieved by use of the present invention.

TABLE 3
Titer of integrated viral complex based vaccine
after prolonged refrigerated storage

| | PFU per vial (×10⁶) | | |
|---|---|---|---|
| | after storage at +4 degree^{s} C. | | |
| Vaccine batch | Initial | 7 months | 12 months |
| CVI988 MD IC-002 | 456 | 472 | 415 |
| CVI988 MD IC-003 | 347 | 353 | 336 |

### Example 4

### Protection of Integrated Vital Complex Based Vaccine Against VV+MDV Challenge in SPF Chickens

In order to eliminate a possible influence of maternal antibodies an additional vaccination/challenge experiment was conducted using SPF chicks as described hereinabove in methods and materials.

Briefly, two hundred 1 day old chicks from SPF embryonated eggs as described hereinabove were hatched in isolators and divided into 5 equal groups.

Chicks were vaccinated with either 500 PFU (groups 1 and 3) or 1000 PFU (groups 2 and 4) per dose of commercial Cell Associated CVI988 vaccine (Rismavac, Intervet, Holland; groups 3 and 4), or with integrated viral complex CVI988 based vaccine according to the present invention (groups 1 and 2). An unvaccinated group served as a negative control (group 5).

Challenge was carried out 9 days post vaccination and 70 days of post challenge observation were performed as in example 2. Results are summarized in table 4. Percent (%) protection is 100 percent minus the % MD positive.

Protection rates of the integrated viral complex based vaccine were similar to those of the commercial Cell Associated (CA) vaccine at either tested dose. These results confirm that the integrated viral complex based vaccine induce an effective level of immunity in commercial birds. Both vaccines comply with potency test of the Ph Eur—Annex—the requirements of the s third edition of the Mark's Disease Vaccine (Live) [0589], and US 9CFR 113.330.

TABLE 4
Relative protection of integrated viral complex based
vaccine and cell associated vaccines

| | | PFU | # MD | | |
|---|---|---|---|---|---|
| | Vaccine | per | Positive | % MD | % |
| Group | type | Dose | birds/Total | positive | protected |
| 1 | Integrated | 500 | 22/38 | 57.9 | 42.1 |
| | viral | | | | |
| | complex | | | | |
| 2 | Integrated | 1000 | 11/39 | 28.2 | 71.8 |
| | viral | | | | |
| | complex | | | | |
| 3 | Cell | 500 | 23/39 | 58.9 | 41.0 |
| | associated | | | | |
| 4 | Cell | 1000 | 10/37 | 27.0 | 73.0 |
| | associated | | | | |
| 5 | (—) | 0 | 35/37 | 94.6 | 5.4 |
| | control | | | | |

### Example 5

### Comparative Efficacy of 3 Different Vaccine Types Derived From the Same Viral Strain

In order to test the suitability of integrated viral complexes according to the present invention as a substitute for cell free vaccines, an additional vaccination/challenge trial was conducted. Briefly, four hundred, 1-day-old commercial chicks (with maternal antibodies), from the same layer breeder flock hatched in commercial hatchery (Hassollellim Hatcheries, Israel) were raised in positive pressure isolators and divided into four equal groups and vaccinated by IM injection with 1000 PFU per dose of Cell Associated HVT vaccine (group a), or Integrated viral complex based HVT vaccine (group b) or Cell Free HVT vaccine (group c). Group d was injected with diluent only and served as a control group.

Viral challenge and post challenge observation were as described hereinabove. Results, summarized in Table 5, indicate that integrated viral complexes of the present invention offer the efficacy of cell associated preparations together with the simple storage conditions previously associated with cell free vaccines.

TABLE 5
Relative protection from 3 vaccine types

| | HVT Vaccine | PFU/ | # MD Pos./# | % MD | % |
|---|---|---|---|---|---|
| Group | type | Dose | total | pos. | protected |
| a | Cell | 1000 | 52/98 | 53.1 | 46.9 |
| | associated | | | | |
| b | Integrated | 1000 | 49/96 | 51.0 | 49.0 |
| | viral complex | | | | |
| c | Cell free | 1000 | 58/97 | 59.8 | 40.2 |
| d | (—) control | zero | 72/98 | 73.5 | 26.5 |

What is claimed:

1. An integrated viral complex, the complex comprising:
(a) a plurality of intact cell membranes, each of said intact cell membranes belonging to a non-viable cell; and
(b) a plurality of viable virions, a majority of said virions of said plurality of viable virions contained within said intact cell membrane belonging to said plurality of intact cell membranes.

2. The integrated viral complex of claim 1, wherein said virions are DNA virions,

3. The integrated viral complex of claim 2, wherein said DNA virions are double stranded DNA virions,

4. The integrated viral complex of claim 3, wherein said double stranded DNA virions belong to the herpes viruses.

5. The integrated viral complex of claim 4, wherein said herpes virus is Marek's disease virus.

6. A pharmaceutical composition for vaccination, the pharmaceutical composition comprising:
(a) a plurality of intact cell membranes, each of said intact cell membranes belonging to a non-viable cell; and
(b) a plurality of viable virions, a majority of said virions of said plurality of viable virions contained within said intact cell membrane belonging to said plurality of intact cell membranes; and
(c) carriers and excipients.

7. The pharmaceutical composition of claim 6, supplied as an article of manufacture including packaging material and instructions for use.

8. The pharmaceutical composition of claim 6, wherein said virions are DNA virions,

9. The pharmaceutical composition of claim 8, wherein said DNA virions are double stranded DNA virions,

10. The pharmaceutical composition of claim 9, wherein said double stranded DNA virions belong to the herpes viruses.

11. The integrated viral complex of claim 10, wherein said herpes virus is Marek's disease virus.

12. A method for producing integrated viral complexes, the method comprising:
(a) growing a population of individual cells in culture;
(b) infecting said individual cells belonging to said population with an aliquot of viable virions so that a desired viral yield is achieved;
(c) transferring said population of individual cells characterized by said desired viral yield to a storage medium containing a cryoprotectant;
(d) storing said population of individual cells characterized by said desired viral yield at a temperature in the range of (−) 30 to (+) 8 degrees centigrade.

13. The method of claim 12, wherein said infecting employ a viral preparation selected from the group consisting of a cell free preparation and a cell associated preparation.

14. The method of claim 12, wherein said cryoprotectant includes at least one material selected from the group consisting of glycerol, DMSO, and sugars.

15. The method of claim 12, wherein said desired viral yield is in the range of 0.001 to 1 PFU/cell.

16. The method of claim 12, wherein said temperature in the range of (+) 2 to (+) 8 degrees centigrade.

17. The method of claim 12, further comprising:
(e) passaging said individual cells belonging to said population with said desired viral yield as a means of increasing a size of said population.

18. The method of claim 12, further comprising:
(e) reducing a volume of said storage medium so that a desired number of cells per unit volume is achieved.

19. The method of claim 12, further comprising:
(e) drying said population of individual cells.

20. A method of producing a pharmaceutical composition for vaccination, the method comprising:
(a) growing a population of individual cells in culture;
(b) infecting said individual cells belonging to said population with an aliquot of viable virions so that a desired viral yield is achieved;
(c) transferring said population of individual cells characterized by said desired viral yield to a storage medium containing a cryoprotectant;
(d) dividing said population of individual cells characterized by said desired viral yield into dosage portions suited for vaccination of a specified number of subjects; and
(e) storing said dosage portions at a temperature in the range of (−) 30 to (+) 8 degrees centigrade.

21. The method of claim 20, wherein said dosage portions each individually include a number of doses in the range of 1 to 1 million.

22. The method of claim 20, wherein said infecting employ a viral preparation selected from the group consisting of a cell free preparation and a cell associated preparation.

23. The method of claim 20, wherein said cryoprotectant includes at least one material selected from the group consisting of glycerol, DMSO, and sugars.

24. The method of claim 20, wherein said desired viral yield is in the range of 0.001 to 1 PFU/cell.

25. The method of claim 20, wherein said temperature in the range of (+) 2 to (+) 8 degrees centigrade.

26. The method of claim 20, further comprising:
(f) passaging said individual cells belonging to said population with said desired viral yield as a means of increasing a size of said population.

27. The method of claim 20, further comprising:
(f) reducing a volume of said storage medium so that a desired number of cells per unit volume is achieved.

28. The method of claim 20, further comprising:
(f) drying said population of individual cells.

29. A method of vaccination which employs an integrated viral complex, the method comprising administering to a subject at least one dose of an amount of an integrated viral complex sufficient to elicit an active immune response in a subject.

30. The method of claim 29, wherein said subject is a member of an avian species.

31. The method of claim 29, wherein said integrated viral complex includes DNA virions,

32. The method of claim 29, wherein said DNA virions are double stranded DNA virions,

33. The method of claim 32, wherein said double stranded DNA virions belong to the herpes viruses.

34. The method of claim 32, wherein said herpes virus is Marek's disease virus.

35. The method of claim 29, wherein said administration is conducted in ovo.

36. The method of claim 29, wherein said administration is conducted via injection or from 1 day of age.
